(19) United States
(12) Patent Application Publication
(10) Pub. No.: US 2025/0288701 A1
(43) Pub. Date: Sep. 18, 2025

(54) REAGENT, PHARMACEUTICAL COMPOSITION AND METHODS FOR DETECTING, EVALUATING THE PROGRESSION, AND TREATING DISEASES ASSOCIATED WITH B-AMYLOID PLAQUES

(71) Applicants: UNIVERSIDAD DE CHILE, Santiago (CL); PONTIFICIA UNIVERSIDAD CATÓLICA DE CHILE, Santiago (CL)

(72) Inventors: Marcelo KOGAN BOCIAN, Santiago (CL); Francisco MORALES ZAVALA, Santiago (CL); Pedro JARA GUAJARDO, Santiago (CL); Alejandra ALVAREZ ROJAS, Santiago (CL); América CHANDÍA CRISTI, Valparaíso (CL)

(21) Appl. No.: 18/860,138

(22) PCT Filed: Apr. 25, 2022
(86) PCT No.: PCT/CL2022/050040

§ 371 Date: Oct. 25, 2024

(51) Int. Cl. A61K 49/00; A61K 33/242; A61K 47/64; A61K 47/69; A61P 25/28; C07K 14/705

(52) U.S. Cl. CPC A61K 49/0065; A61K 33/242; A61K 47/6425; A61K 47/6929; A61K 49/0056; A61P 25/28; C07K 14/70571

(57) ABSTRACT

The present invention relates to the technical field of nanotechnology and pharmaceutics, particularly, it relates to a reagent and pharmaceutical composition containing gold nanorods conjugated to D1 and Angiopep-2 peptides, and methods for detecting, diagnosing, evaluating the progression, and treating diseases related to β-amyloid plaques, such as Alzheimer's disease. Surprisingly, with the reagent and pharmaceutical composition of the present invention, a dose of gold per kg of body weight well below those previously reported is required, hence their applications are more economical, in addition to decreasing the likelihood of toxic effects, thanks to the low doses required to exert their function.

### TECHNICAL FIELD

The present invention relates to the technical field of nanotechnology and pharmaceutics, particularly, it relates to a reagent and a pharmaceutical composition containing gold nanorods conjugated to D1 and Angiopep-2 peptides, and methods for detecting, evaluating the progression, diagnosing, and treating diseases associated with β-amyloid plaques.

### BACKGROUND

In the field of medicine, multifunctional nanoparticles are of great interest, especially for the diagnosis and treatment of various diseases and also as drug delivery systems. Particularly, multifunctional gold nanoparticles may be used in imaging and phototherapy, integrating both in a single system for diagnosis and treatment, so they can be considered as a theragnostic agent.

Gold nanoparticles (GNPs) have been used at the preclinical level as a contrast agent for computed tomography (CT) due to their high X-ray attenuation properties. Typically, concentrations of GNPs used as a CT contrast agent are of the order of mg of gold per kg of body weight (Kim J Y, et al. (2015). Theragnostics, 5(10), 1098-1114; Boote E. et al. (2010). Acad Radiol, 17, 410-417). As GNPs are easily functionalized, they have been modified to target the contrast agent specifically in the tissue of interest, improving the limitations associated with CT imaging. Thus, the use of GNPs as a CT contrast agent provides a rapid and noninvasive system for in vivo diagnostics.

On the other hand, recent studies indicate that GNPs can reach the central nervous system (CNS) by crossing the blood-brain barrier, so they could be used as a theragnostic agent for neurodegenerative diseases (Velasco-Aguirre C. et al. (2015). Int. J. Nanomed., 10, 4919-4936; Velasco-Aguirre C. et al. (2017). Nanomedicine (Lond.), 12, 2503-2517; Jara-Guajardo P. et al. (2020). Nanomaterials, 10, 690). This is of particular importance since early detection of neurodegenerative diseases is crucial to attenuate disease progression.

Alzheimer's disease (AD) is the most common dementia in the elderly. While the pathological features and molecular mechanisms of AD are known, early diagnosis and the development of effective therapies remain major scientific challenges.

The β-amyloid (Aβ) peptide is a key etiological agent in AD, responsible for neurotoxicity, neuronal loss, and subsequent cognitive decline. In vivo detection of soluble Aβ species is particularly relevant because these are highly toxic and prevalent in the early stages of the disease. The accumulation of Aβ in the brain (Aβ plaque formation) is a reliable pathological biomarker of AD, and is thought to trigger the synaptic dysfunction and neurodegeneration associated with the cognitive decline suffered by patients suffering from AD (Karran E, et al. (2011). Nat. Rev. Drug Discovery, 10, 698-712; Ballard C, et al. (2011). Lancet, 377, 1019-1031).

Several solutions for early detection of Aβ plaques in the brain have been described in the prior art. Such is the case of patent document U.S. Pat. No. 10,258,699, which refers to a method for detecting amyloid deposits by imaging, using an agent containing the Angiopep-2 peptide, a linker, and a group suitable for detection by imaging, wherein said suitable group can be FITC, Cy5.5, a fluorescent protein, a radioactive isotope, iron oxide particles, gold nanoparticles, among others. Particularly, it mentions that gold nanoparticles are used for the detection of amyloid deposits in the brain by computed tomography. This document mentions that the claimed method may be useful for detecting amyloid-related diseases such as AD, among others. This document can be considered as general prior art related to the invention.

The scientific article by Morales-Zavala et al. (2017). Nanomed: Nanotechnol. Biol. Med., 13, 2341-2350, discloses a study of PEG-stabilized gold nanorods conjugated to D1 and Angiopep-2 peptides for in vitro evaluation of neuronal viability and cytotoxicity of the nanorods in a primary culture of hippocampal neurons, and in vivo evaluation of the effects of said conjugated nanorods on Aβ peptide aggregation. The study model was done in transgenic Caenorhabditis elegans expressing the Aβ₁₋₄₂ peptide in muscle cells. The expression of this peptide causes alterations in the locomotor apparatus of nematodes. Although C. elegans is a good initial in vivo model to evaluate drugs or other reagents prior to experimentation in vertebrate animal models, it does not provide certainty that the results observed in said model can be replicated in higher animal models.

On the other hand, the scientific article by Velasco-Aguirre et al. (2017) Nanomedicine (Lond.), 12(20), 2503-2517, discloses a study to increase the delivery of gold nanorods in vivo to the central nervous system of rats. The nanorods were conjugated to the Angiopep-2 peptide that allows crossing the blood-brain barrier. The cytotoxicity of these nanorods conjugated in vitro and their biodistribution were evaluated by STEM imaging and ex vivo fluorescence imaging. Sprague-Dawley rats were injected with 1.86 mg gold per kg of body weight intravenously, they were sacrificed, their brains removed, and subsequently the nanorods were visualized in said organ. This article does not use an animal model associated with neurodegenerative diseases, such as AD.

Although the disclosures of the prior art show an approach to the problem of finding alternatives for the use of gold nanoparticles conjugated to peptides of interest to target these conjugates to biological targets of importance in the diagnosis and/or therapy of neurodegenerative diseases, such as AD, there is no clear and precise alternative of the conditions under which these nanoparticles have to be used for the detection and treatment of amyloid plaques in vivo.

### SUMMARY OF THE INVENTION

The present invention relates to a reagent and a pharmaceutical composition containing gold nanorods conjugated to D1 and Angiopep-2 peptides, and methods for detecting, evaluating the progression, diagnosing, and treating diseases related to β-amyloid plaques, wherein a dose of gold per kg of body weight well below those reported in the prior art is used.

A first object of the present invention is a reagent that binds to β-amyloid plaques in a subject, comprising gold nanorods conjugated to D1 and Angiopep-2 peptides, including a dose of said gold nanorods conjugated to the D1 and Angiopep-2 peptides between about 8.6 and 860 μg of Au per kg of body weight of the subject, and a pharmaceutically acceptable vehicle. In a preferred embodiment of the reagent, the reagent is formulated for intranasal or intravenous administration.

In a preferred embodiment of the reagent, the gold nanorods are conjugated to the D1 and Angiopep-2 peptides at a molar ratio of gold nanorods:peptides of 1:600. In another preferred embodiment, the D1 and Angiopep-2 peptides are at a molar ratio of 1:9. In another preferred embodiment, the conjugated gold nanorods have all their dimensions in the nanometer range and have a length/width aspect ratio between 1 and 5.

A second object of the present invention is a method for targeting a reagent including gold nanorods conjugated to D1 and Angiopep-2 peptides to β-amyloid plaques in a subject, comprising contacting said reagent at a dose between 8.6 and 860 μg of Au per kg of body weight of the subject with a tissue or organ of the subject, and allowing said reagent to bind to the β-amyloid plaques present in the tissue or organ. Preferably, the reagent binds to β-amyloid plaques present in the brain. The reagent used in this method preferably comprises any one or a combination of the technical specifications described previously in the preferred embodiments of the reagent.

A third object of the present invention is a method for detecting β-amyloid plaques in a subject, using a reagent including gold nanorods conjugated to D1 and Angiopep-2 peptides, comprising the steps of: a) administering to the subject a dose of said reagent between about 8.6 to 860 μg of Au per kg of body weight of the subject; b) allowing said reagent to bind to the β-amyloid plaques present in a tissue or organ of said subject, preferably in the brain; and c) subjecting the subject to an imaging study to detect β-amyloid plaques to which said reagent binds. In a preferred embodiment, the reagent is administered to the subject intravenously or nasally. In a preferred embodiment of the method for detecting β-amyloid plaques, the imaging study is by computed tomography.

A fourth object of the present invention is a method for evaluating the progression of the accumulation of β-amyloid plaque overtime in a subject, using a reagent including gold nanorods conjugated to D1 and Angiopep-2 peptides, comprising the steps of: a) determining a reference value of the number of β-amyloid plaque present in a tissue or organ of said subject at a time 0; b) determining a value of the number of β-amyloid plaque present in the tissue or organ of said subject at a time n; and c) comparing the value of the number of β-amyloid plaque at the time n with the reference value at the time 0; and d) if the value of the number of β-amyloid plaque at the time n is greater than the reference value at the time 0, it is an indication of the progression of the accumulation of β-amyloid plaque over time in the subject.

Determining the values at the time 0 and the time n includes the steps of: i) administering to the subject a dose of said reagent between about 8.6 and 860 μg of Au per kg of body weight of the subject; ii) allowing said reagent to bind to the β-amyloid plaques present in a tissue or organ of said subject; and iii) subjecting the subject to an imaging study to quantify the β-amyloid plaques to which said reagent binds. In a preferred embodiment, the reagent is administered to the subject intravenously or nasally. In another preferred embodiment, the organ in which the presence of β-amyloid plaques is determined is the brain. In a preferred embodiment of the method for evaluating the accumulation of β-amyloid plaques, the imaging study is by computed tomography.

A fifth object of the present invention is a pharmaceutical composition for treating neurodegenerative diseases associated with β-amyloid plaques in a subject, including gold nanorods conjugated to D1 and Angiopep-2 peptides, said pharmaceutical composition comprising a dose of said gold nanorods conjugated to D1 and Angiopep-2 peptides between about 8.6 and 860 μg of Au per kg of body weight of the subject, and a pharmaceutically acceptable vehicle. In a preferred embodiment of the pharmaceutical composition, the same is formulated for intranasal or intravenous administration.

In a preferred embodiment of the pharmaceutical composition, the gold nanorods are conjugated to the D1 and Angiopep-2 peptides at a molar ratio of gold nanorods:peptides of 1:600. In another preferred embodiment, the D1 and Angiopep-2 peptides are at a molar ratio of 1:9. In another preferred embodiment, the conjugated gold nanorods have all their dimensions in the nanometer range and have a length/width aspect ratio between 1 and 5.

In another preferred embodiment of the pharmaceutical composition, the neurodegenerative disease associated with β-amyloid plaques is Alzheimer's disease.

A sixth object of the present invention is a method for treating diseases associated with β-amyloid plaques in a subject using a pharmaceutical composition including gold nanorods conjugated to the D1 and Angiopep-2 peptides, comprising administering to the subject a dose of said pharmaceutical composition between about 8.6 and 860 μg of Au per kg of body weight of the subject. In a preferred embodiment of the pharmaceutical composition, the same is formulated for intranasal or intravenous administration. The pharmaceutical composition used in this method of treatment includes, preferably, any one or a combination of the technical specifications described previously in the preferred embodiments of the pharmaceutical composition.

Preferably, the neurodegenerative disease related to or associated with β-amyloid plaques is Alzheimer's disease.

### BRIEF DESCRIPTION OF THE FIGURES

FIG. 1. STEM images of the conjugated nanorods. FIG. 1A: GNRs-PEGs.

FIG. 1B: GNRs-D1/Ang2. The bar is equal to 500 nm.

FIG. 2. Distribution histogram showing the length/width of GNR-D1/Ang2.

FIG. 3. UV-Visible spectrum of GNRs-PEGs and GNRs-D1/Ang2.

FIG. 4. Cell population positive for incorporation of GNRs-D1 and GNRs-D1/Ang2, which was evaluated by flow cytometry. The bEnd.3 cells were incubated for 2 h with the different modified GNRs at a concentration of 0.05 nM. The GNRs were labeled with Alexa 647 for their detection. One group of cells was incubated at 4° C. for 2 hours with GNRs (n=5) and another group was pretreated with CPZ for 15 minutes at a concentration of 50 μM (n=6). Turkey's multiple comparisons test (**** p<0.0001).

FIG. 5. Transmission electron microscopy (TEM) of bEnd.3 cells incubated with GNRs-D1/Ang2 [0.05 nM] for 24 hours. Yellow arrows indicate GNRs. Bar=50 and 100 nm.

FIG. 6. Percentage of the gold dose that reached the brain. A total of 100 μl [10 nM] of GNRs-PEG or GNRs-D1/Ang2 were injected intravenously (i.v.). The animals were sacrificed at 15 min. Gold quantification was performed by neutron activation (Chilean Nuclear Energy Commission, CCHEN), n=4. Mann-Whitney test, p<0.05.

FIG. 7. Percentage of the gold dose that reached the liver. 100 μl (10 nM) of GNRs-PEGs or GNRs-D1/Ang2 were injected into the tail vein of APPswe/PSEN1dE9 (TG) transgenic mice (n=4). The animals were sacrificed at 15 min. Gold quantification was performed by neutron activation.

FIG. 8. STEM dark-field imaging of brain sections in hippocampal regions for the detection of GNRs-D1/Ang2. A total of 100 μl [10 nM] of GNRs-D1/Ang2 was injected i.v. Yellow arrows indicate the GNRs. Bar=200 nm.

FIG. 9. Immunohistochemistry fluorescence imaging of brain slices from transgenic mouse models of Alzheimer's disease after 15 min of the i.v. injection of 100 μl of GNRs-D1/Ang2-Alexa Fluor 647 [10 nM]. The amyloid plaques are seen in green. The control was injected with GNRs-PEGs [10 nM].

FIG. 10. Percentage of signal overlap GNRs with the amyloid plaques from the in vivo experiment presented in FIG. 5. Mann-Whitney test, p<0.05.

FIG. 11. Effects of GNRs-D1/Ang2 on amyloid load in the brain cortex. Animals treated with GNRs-D1/Ang2 (15 administrations in a 30-day period, 100 μl [10 nM], intraperitoneal (i.p.), n=4) showed a significant reduction in amyloid load evaluated by thioflavin S, compared to the control group treated with GNRs-Ang2. FIG. 11A: Representative image of cortex brain slices of animals treated with the GNRs-Ang2 and GNRs-D1/Ang2 nanosystems. FIG. 11B: Quantification of the area occupied by amyloid plaques in the GNRs-Ang2 and GNRs-D1/Ang groups, expressed as a percentage of the occupied area with respect to the total image area. Mann-Whitney test, p<0.0001.

FIG. 12. Effect of GNRs-D1/Ang2 on GFAP intensity in the brain cortex. Animals treated with GNRs-D1/Ang2 (15 administrations in a 30-day period, 100 μL [10 nM], i.p., n=4) showed a significant reduction in GFAP signal, as evaluated by immunofluorescence, compared to the control group treated with GNRs-Ang2. FIG. 12A: Representative images of cortex brain slices of animals treated with the GNRs-Ang2 and GNRs-D1/Ang2 nanosystems. FIG. 12B: Quantification of the GFAP signal intensity. Mann-Whitney test, p<0.0001.

FIG. 13: Images of the skull by micro-CT of transgenic APPswe/PSEN1dE9 mice treated for one month (15 administrations) with 100 μL of GNRs-D1/Ang2 or GNRs-Ang2 10 nM. FIG. 13A: Sagittal section of a transgenic mouse treated with GNRs-Ang2. FIG. 13B: Sagittal section of the skull of a transgenic mouse treated with GNRs-D1/Ang2. FIG. 13C: Frontal section of the skull of a transgenic mouse treated with GNRs-Ang2. FIG. 13D: Frontal section of the skull of a transgenic mouse treated with GNRs-D1/Ang2. The white arrows indicate accumulation of gold nanoparticles in the amyloid aggregates. The red arrows indicate accumulation of gold nanoparticles in the circulation.

FIG. 14. Images of the skull by micro-CT of transgenic APPswe/PSEN1dE9 mice (TG) or healthy C57BL/6 mice (WT) treated with a single dose of GNRs-D1/Ang2 (10 nM) administered i.v. Sagittal sections (upper panels) and frontal sections (lower panels) of mice treated with GNRs-D1/Ang2 are shown in the figure. The white arrows indicate the signal associated with the presence of GNRs.

FIG. 15. Schematic of the evaluation of cell penetration (in vitro), brain delivery (in vivo), amyloid plaques attachment (ex vivo), and in vivo detection of GNRs-D1/Ang2 by micro-CT.

FIG. 16. Intensity histograms of Micro-CT of a transgenic APPswe/PSEN1dE9 mouse (TG) treated with a single dose of GNRs-D1/Ang2 (1 nM). FIG. 16A: Pre-treatment stage. FIG. 16B: Post-treatment stage. FIG. 16C: Mounted images of histograms of both stages. FIG. 16D. Rescaling for area of interest.

FIG. 17. Set of comparative images between the different stages of GNRs-D1/Ang2 administration and stages of image processing for the 1 nM dose. The upper part shows the pre-treatment (left) and post-treatment (right) unprocessed images. The lower part shows the pre-treatment (left) and post-treatment (right) processed images.

FIG. 18. Intensity histograms of Micro-CT of a transgenic APPswe/PSEN1dE9 mouse (TG) treated with a single dose of GNRs-D1/Ang2 (10 nM). FIG. 18A: Pre-treatment stage. FIG. 18B: Post-treatment stage. FIG. 18C: Mounted image of histograms of both stages. FIG. 18D. Rescaling for area of interest.

FIG. 19. Set of comparative images between the different stages of GNRs-D1/Ang2 administration and stages of image processing for the 10 nM dose. The upper part shows the pre-treatment (left) and post-treatment (right) unprocessed images. The lower part shows the pre-treatment (left) and post-treatment (right) processed images.

### DETAILED DESCRIPTION OF THE INVENTION

The present invention comprises a reagent capable of binding to β-amyloid (Aβ) plaques in a subject, wherein said reagent contains gold nanorods (GNRs) conjugated to D1 and Angiopep-2 peptides (GNRs-D1/Ang2). These nanorods can cross the blood-brain barrier and specifically target Aβ plaques present in the brain of a subject.

Surprisingly, with the reagent of the present invention a dose well below those used in the prior art, of the order of μg of Au per kg of body weight, is required for its use, for example, as a contrast agent, preferably for the visualization of Aβ by computed tomography (CT). This allows the applications given to this reagent to be more economical, as well as decreasing the likelihood of toxic effects, due to the low doses required to exert its function.

The reagent of the present invention may be used in a method for peptide-conjugated gold nanorods to specifically target the Aβ plaques and bind to them within a few minutes after administration of said reagent. The method of the present invention may also be used to detect Aβ plaques and visualize them by means of imaging techniques, and after analyzing the results of the imaging study it is possible to evaluate the accumulation of said plaques over time, diagnose whether a subject or individual suffers from a neurodegenerative disease associated with Aβ plaques, and see the progression of the disease based on the accumulation of said plaques. Surprisingly, it is possible to early detect Aβ plaques in a subject, even using the low dose or concentration of the conjugated gold nanorods disclosed herein. Therefore, the reagent described herein has a high sensitivity, higher than that known to date.

Additionally, gold nanorods conjugated to D1 and Angiopep-2 peptides may be part of a pharmaceutical composition useful for treating neurodegenerative diseases related to the accumulation of Aβ plaques.

All technical and scientific terms used to describe the present invention have the same meaning as understood to a person with basic knowledge in the technical field in question. However, in order to define the scope of the invention more clearly, the following list of the principal terminology used in this description is included.

The term “reagent” shall be understood as a formulation comprising some type of molecule capable of reacting, interacting, or binding to another molecule outside the formulation. For example, the reagent of the present invention contains nanorods that are capable of binding to Aβ plaques. The reagents of the present invention are preferably oriented to be used in methods for the detection or visualization of Aβ plaques present in a tissue or organ of a subject or individual by imaging techniques. When used in said application, the reagent may also be referred to as a “contrast agent”, “contrast medium”, “contrast material”, or the like.

The term “nanorods” shall be understood as particles having an elongated cylindrical morphology or in the shape of a rod, bar, or tube, having all their dimensions in the nanometer range, typically with dimensions less than 100 nm. Generally, nanorods have an aspect ratio of length divided by width between 1 and 5, but are not limited to said range.

The term “conjugated nanorods” shall be understood as nanorods containing molecules bound to their surface by a chemical or physical interaction, where said molecules may be biological molecules such as peptides, lipids, carbohydrates, nucleic acids, or other compounds such as drugs, radioactive isotopes, fluorophores, etc. Said binding may be mediated by connecting molecules or “linkers”. For purposes of the present invention, the terms “conjugated nanorod”, “conjugated gold nanorod”, “nanosystems”, or “nanoconjugates” will be used interchangeably to refer to gold nanorods conjugated to the D1 and Angiopep-2 peptides.

The term “detect” shall be understood as discovering or finding the existence of something that was not previously detectable. For purposes of the present invention, detection would be finding Aβ plaques in a tissue or organ of a subject. When detection is performed by imaging techniques, the Aβ plaques can be visualized using the reagent or contrast agent of the present invention.

From the detection or visualization of that which was not previously detectable in a subject, in this case, the detection of Aβ plaques preferably by an imaging technique, the results of said detection, which may be qualitative and/or quantitative, may be collected and analyzed to diagnose the presence or absence of a disease, the stage at which it is found, and its progression. For purposes of the present invention, the diagnosis would relate to a neurodegenerative disease associated with the accumulation of Aβ plaques.

The term “subject” shall be understood as any mammal, such as humans, mice, rats, rabbits, primates, cats, dogs, among others, without being limited to those mentioned herein. The term “subject” shall be understood as synonymous with the term “individual” and shall be used interchangeably in the present description.

The term “treatment” shall be understood as a set or means employed with a therapeutic purpose on a subject who has developed or is in the early stages of developing a disease, syndrome, or condition. The treatment may be a treatment of the symptomatology of the subject with the purpose of alleviating its symptoms, it may refer to a treatment of the cause originating the disease, syndrome, or condition, with the purpose of reversing, slowing, or halting the progression of said disease, syndrome, or condition. Therefore, the pharmaceutical composition of the present invention, as well as the methods described herein, may be used, for example, as methods of therapeutic treatment for a specific period of time or on a chronic basis, as required by the subject. The term “treatment” also includes those preventive treatments comprising treating subjects who are at risk of developing a disease, syndrome, or condition, in order to reduce said risk.

The terms “about” or “approximately”, used interchangeably throughout the present description, shall be understood as the value or range of a parameter which includes a standard deviation of error according to the method or apparatus used to determine said value or range, and which is within the acceptable tolerance or statistically significant range for the value of said parameter. Said statistically significant range may be, for example, within 30%, 20%, 10%, or 5% of the indicated value or range. That is, the mentioned values and ranges are not and need not be exact, and may be approximate, either equal, lower, or greater. The term “between” when referring to ranges, is to be understood as any intermediate value between around the lower value and around the upper value mentioned.

A first object of the present invention corresponds to a reagent including gold nanorods conjugated to the D1 and Angiopep-2 peptides at a concentration surprisingly lower than the concentration used in the prior art, and a pharmaceutically acceptable vehicle. The conjugated gold nanorods bind to Aβ plaques in a tissue or organ of an individual, allowing them to be visualized, detected, or even disaggregated. The Angiopep-2 peptide (whose amino acid sequence is TFFYGGSRGKRNNFKTEEY (SEQ ID NO:1)) favors the conjugated nanorods to cross the blood-brain barrier. On the other hand, the D1 peptide (whose amino acid sequence is H-qshyrhispaqv-OH (SEQ ID NO:2)) recognizes, disaggregates Aβ plaques, and reduces the toxicity of said plaques. Therefore, the reagent of the present invention containing gold nanorods conjugated to the D1 and Angiopep-2 peptides, may be considered as a theragnostic agent, since it may be used both for diagnosing neurodegenerative diseases associated with Aβ plaques and for treating said diseases.

Particularly, the nanorods of the present invention are modified with polyethylene glycol (PEG) to increase the biocompatibility of the nanorods, maintain their colloidal stability, and act as a bridge or connector between the nanorod and the peptides to be conjugated. Thiol terminated polyethylene glycol (containing a-SH thiol group) may be used at one end, and at its terminal end it may contain a methoxy group (HS-PEG-OMe) or a carboxylic group (HS-PEG-COOH). Both types of PEGs have a molecular weight of 5000 kDa. Therefore, the conjugated nanorods of the present invention may also be abbreviated as GNRs-PEG-D1/Angiopep-2; GNRs-PEG-D1/Ang2; GNRs-D1/Angiopep-2 or GNRs-D1/Ang2, interchangeably.

The gold nanorods have an average length of 40±5 nm, and an average width of 11±1 nm, observed with electron microscopy (STEM). On the other hand, GNR-D1/Angiopep-2 have an estimated hydrodynamic diameter of less than 150 nm.

The molar ratio between the D1 and Angiopep-2 peptides present in the nanorods is about 1:9, respectively. In turn, the molar ratio between the gold nanorods and the peptides is about 1:600.

The reagent containing the conjugated nanorods is used in the methods of the present invention at a dose of less than 860 μg approximately of Au per kg of body weight of the subject (μg of Au/kg), preferably about 86 μg of Au/kg. Preferably, the reagent containing the conjugated nanorods is used in the methods of the present invention at a dose between 8.6 and 860 μg approximately of Au per kg of body weight of the subject, more preferably about 86 μg of Au per kg of body weight of the subject. These doses are about 100 times lower than those reported in the prior art for their use as CT contrast agents.

The reagent of the present invention also comprises a pharmaceutically acceptable vehicle. Said vehicle shall be understood in a broad manner, and may refer to an excipient, a diluent, a stabilizer, an adjuvant, or a mixture thereof, or other compound of interest. These vehicles are known in the prior art, such as those reported, for example, in Allen L, Popovich N, Ansel H. (2011). Ansel's Pharmaceutical Dosage Forms and Drug Delivery Systems. 9th edition. Lippincott Williams & Wilkins, and Rowe R, Sheskey P, Quinn M. (2009). Handbook of Pharmaceutical Excipients. 6th edition. Pharmaceutical Press, without being limited to these.

In a preferred embodiment of the invention, the reagent is formulated for its administration by injections and/or parenterally, for example, intravenously, intra-arterially, intramuscularly, intraperitoneally, or directly into organs, without being limited to these routes. The reagent of the present invention may also be administered to the subject by intranasal route. For this reason, the reagent is preferably formulated in liquid form as an aqueous or oily suspension, which may include a vehicle, excipient, dispersants, among others. The reagent may also be formulated in solid or semi-solid form. The specific agents and compounds for each of these functions are obvious to a person skilled in the art and can be found in multiple publications such as those previously mentioned.

A second object of the present invention corresponds to a method for targeting the above-described reagent to Aβ plaques in a subject, comprising contacting the reagent at a dose of less than 860 μg approximately of Au per kg of body weight of the subject (μg of Au/kg) with a tissue or organ of the subject, preferably about 86 μg of Au/kg. Preferably, the reagent contacts the tissue or organ at a dose between 8.6 and 860 μg approximately of Au per kg of body weight of the subject, more preferably about 86 μg of Au per kg of body weight of the subject.

In a preferred embodiment of this second object of the present invention, the reagent is brought into contact with the tissue or organ, preferably the brain of the subject, through the administration of said reagent intranasally, intravenously, or the like. The time required for the reagent to reach the brain and bind to Aβ plaques depends on multiple factors such as the general condition of the subject, among others. For example, it may be approximately 60 minutes, preferably less than 60 minutes, more preferably less than 30 minutes, even more preferably less than 15 minutes. This time could be even less depending on the condition of the subject.

A third object of the present invention corresponds to a method for detecting Aβ plaques in a subject, using the previously described reagent of the present invention. For this purpose, it is first required to administer to the subject a dose of said reagent of less than 860 μg approximately of Au per kg of body weight of the subject (μg of Au/kg), preferably about 86 μg of Au/kg. Preferably, the reagent containing the conjugated nanorods is used in this method at a dose between 8.6 and 860 μg approximately of Au per kg of body weight of the subject, more preferably about 86 μg of Au per kg of body weight of the subject. Then, an adequate time is allowed for said reagent to bind to Aβ plaques present in a tissue or organ of said subject. Finally, the subject is subjected to an imaging study to detect the Aβ plaques that bound to said reagent.

In a preferred embodiment, the route of administration of the reagent is any of the aforementioned routes, preferably intranasally or intravenously. After having administered the reagent to the subject, an adequate time is allowed for the conjugated nanorods to bind to the Aβ plaques. Said time depends on multiple factors such as the general condition of the subject, among others. For example, it may be approximately 60 minutes, preferably less than 60 minutes, more preferably less than 30 minutes, even more preferably less than 15 minutes. From the tests performed it was observed that, with the reagent of the present invention, it was possible to detect Aβ plaques in the brain at around 15 minutes, the time at which the observation was made. However, this does not mean that said plaques could not be detected in a shorter time.

After this period, the subject undergoes an imaging study to detect and visualize the conjugated nanorods that are bound to the Aβ plaques. The study may be performed by X-Ray, Computed Tomography (CT), or other similar methodologies known in the prior art. In a preferred embodiment of the invention, the concentration of the reagent is optimized for detecting the Aβ plaques by CT.

By using this detection method, it may be determined whether the subject has presence or absence of Aβ plaques in a particular tissue or organ, the number of Aβ plaques, and even determine the stage of the disease.

A fourth object of the present invention corresponds to a method to evaluate the progression of the accumulation of β-amyloid plaques over time in a subject, using the previously described reagent of the present invention. For this purpose, first, a reference value of the number of β-amyloid plaques present in a tissue or organ of said subject at a time 0 is determined. This time 0 corresponds to the first analysis or study of the presence of β-amyloid plaques in the subject. To determine said reference value, the steps or stages of the previously described method for detecting β-amyloid plaques are preferably performed. Then, a value of the number of β-amyloid plaques present in the tissue or organ of said subject is determined at a time n. This time n is any time after the first analysis or study of the presence of β-amyloid plaques in the subject. The determination of this value is preferably performed by the steps or stages of the previously described method for detecting β-amyloid plaques. After obtaining the values of the number of β-amyloid plaques present at the time 0 and time n, they are compared and, if the value of the number of β-amyloid plaques at the time n is greater than the reference value at the time 0, it is an indication of the progression of the accumulation of β-amyloid plaque over time in the subject.

This method is particularly useful for making measurements of the accumulation of β-amyloid plaque over time, which may be correlated with the progression of a disease associated with Aβ plaques. For example, a subject can be tested at an early age to detect the presence or absence of Aβ plaques, without said subject having had any symptomatic manifestation of the effects produced by the accumulation of said plaques. If Aβ plaques are detected, it may be monitored over time which could be useful in devising a strategy to slow the rate at which Aβ plaques accumulate and thus delay the onset of symptoms associated with a neurodegenerative disease, e.g., Alzheimer's disease.

A fifth object of the present invention corresponds to a pharmaceutical composition useful for treating neurodegenerative diseases associated with Aβ plaques, comprising gold nanorods conjugated to the D1 and Angiopep-2 peptides, including a single dose between about 8.6 and 860 μg of Au per kg of body weight of the subject (μg of Au/kg), and a pharmaceutically acceptable vehicle. The nanorods include one or more of the previously described technical features. In a preferred embodiment, the neurodegenerative disease associated with Aβ plaques is Alzheimer's disease (AD), a disease for which the present invention is optimized, but is not limited thereto.

Ordinarily, a person skilled in the art will understand that the reagent and the pharmaceutical composition of the present invention could have different formulations for different uses. However, the pharmaceutically acceptable vehicles may be any of those previously mentioned, without being limited thereto.

A sixth object of the present invention corresponds to a method for treating diseases associated with Aβ plaques using the previously described pharmaceutical composition. Thanks to the properties of the conjugated nanorods described in the present invention, these may be considered as theragnostic agents, i.e., they may be included both in a reagent useful for diagnosing neurodegenerative diseases associated with Aβ plaques and in a pharmaceutical composition useful for treating said disease.

This method of treatment comprises administering to the subject a dose of said pharmaceutical composition between about 8.6 and 860 μg of Au per kg of body weight of the subject (μg of Au/kg), preferably intranasally or intravenously, as a single dose or multiple doses. However, it is not limited to these routes of administration since the pharmaceutical composition of the present invention may be formulated for its administration by injections and/or parenterally, for example, intra-arterially, intramuscularly, intraperitoneally, or directly to the organs, without being limited to the aforementioned routes. For this reason, the pharmaceutical composition is preferably formulated in liquid form, such as an aqueous or oily suspension, which may include a vehicle, excipient, dispersants, among others. The reagent may also be formulated in solid or semi-solid form. The specific agents and compounds for each of these functions are obvious to a person skilled in the art and can be found in multiple publications such as those mentioned above.

The pharmaceutical composition may be administered to the subject on a one dose per day, alternate day, or every second day schedule, but may be modified depending on the physiological condition of the subject.

In a preferred embodiment of the invention, any of the methods of treatment, including preventing, reducing, or delaying the progression, are focused on Alzheimer's disease, but could be adapted to treat other neurodegenerative diseases such as, for example, Huntington's disease.

The pharmaceutical composition may also be used in methods for preventing, reducing, or reversing the progression, slowing or delaying a neurodegenerative disease associated with the accumulation of Aβ plaques; methods for preventing, reducing, or alleviating dementia symptoms of said disease; or methods for improving, reducing, slowing or delaying cognitive impairment of a subject caused by said disease. For example, since the conjugated nanorods of the present invention allow the early detection and diagnosis of the appearance and/or accumulation of Aβ plaques in a subject, even without exhibiting symptomatology associated with the disease that the Aβ plaques produce, treatment can be initiated early to prevent, delay, or reduce the risk of developing said disease.

The following examples are intended to illustrate the invention and its preferred embodiments, but under no circumstances should they be considered to restrict the scope of the invention, which will be defined by the content of the claims included hereto.

### EXAMPLES OF EMBODIMENTS

### Example 1. Synthesis of Gold Nanorods Conjugated to D1 and Angiopep-2 Peptides, Hereinafter GNRs-D1/Ang2

### Materials

The compounds HAuCl₄, cetyltrimethylammonium bromide (CTAB), sodium borohydride, ascorbic acid, AgNO₃, and sulfo-NHS were obtained from Sigma Chemical Co, St Louis, MO, USA. The compounds HS-PEG-OMe and HS-PEG-COOH, both of 5000 kDa, were obtained from JenKem Technology, TX, USA.

### Synthesis of Gold Nanorods (GNRs)

GNRs were synthesized using the seed-mediated growth approach (Adura C, et al. (2013), ACS Appl. Mater. Interfaces, 5, 4076-4085; Perets N, et al. (2019), Nano Lett., 19, 3422-3431). In the first step, a seed solution was prepared to reduce 250 μl of HAuCl₄ in 9.75 ml of 0.1 M cetyltrimethylammonium bromide (CTAB) and cold-prepared sodium borohydride (600 μl, 0.01 M). The seeds were kept at 27° C. for two hours before use. Next, 55 μl of 0.1 M ascorbic acid was added to a growth solution containing 75 μl of 0.01 M AgNO₃, 9.5 ml of 0.1 M CTAB and 500 μl of 0.01 M HAuCl₄. Then, 250 μl of 0.1 M HCl and finally 12 μl of the previously prepared seed solution were added. The solution was incubated for 10 min at 27° C. before centrifugation at 5900 g for 15 min. After centrifugation, the supernatant was removed, and the pellet was resuspended in Milli-Q water.

### GNRs PEGylation and D1 and Ang2 Peptides Conjugation

PEGylation was performed as described in Huang et al. (2010) ACS Nano, 4, 5887-5896. D1 and Ang2 peptides were synthesized and characterized as described in Velasco-Aguirre C. et al. (2017), Nanomedicine (Lond.), 12, 2503-2517; Jara-Guajardo P. et al. (2020), Nanomaterials, 10, 690.

Briefly, D1 and Ang2 peptides and were conjugated to GNRs in a three-step procedure:
1. 50 μl of 1 mM HS-PEG-OMe (molecular weight 5000 kDa) was added to 10 ml of 1 nM GNRs and the solution mixture was stirred for 10 min. The pegylated GNRs (GNRs-PEG) were centrifuged at 20 800 g for 10 min and resuspended in 10 ml of Milli-Q water.
2. 300 μl of 1 mM HS-PEG-COOH (molecular weight 5000 kDa) was added and the solution was stirred for 1 h. The solution was centrifuged twice at 20 800 g for 10 min and resuspended in 100 μl of MES buffer pH 5.5.
3. 0.2 mg EDC and 0.5 mg sulfo-NHS were incubated with the GNRs-PEG for 15 min to activate the carboxyl groups on the GNRs. Excess EDC/sulfo-NHS was removed by centrifugation at 20 800 g for 10 min, and the activated GNRs-PEG were mixed with the D1 and Ang2 peptides in PBS buffer (pH 7.4) at a molar ratio of 1:600 GNRs:peptides. The solution was gently shaken for 2 h at room temperature (RT) and then stored at 4° C. Before use, the GNRs-D1/Ang2 were washed by centrifugation to eliminate the free reactants.

### Example 2. Characterization of GNRs-D1/Ang2

### Morphology and Size of GNRs-D1/Ang2

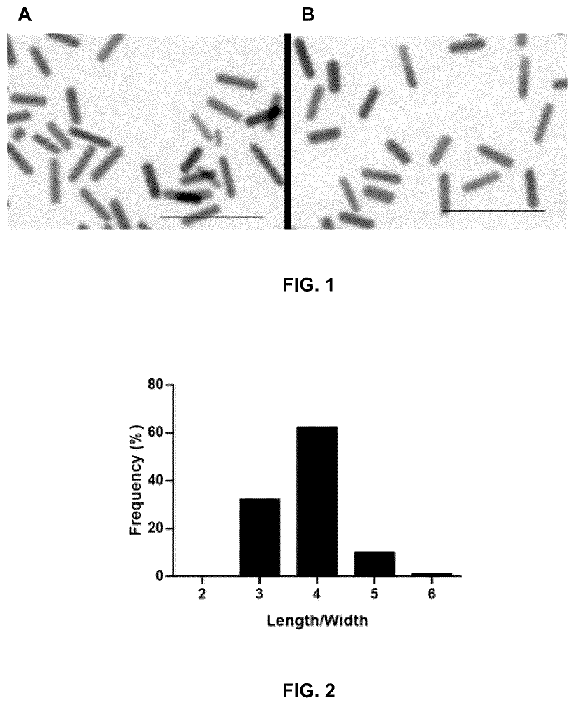

The synthesized GNRs-CTAB, GNRs-PEG, GNRs-Ang2, GNRs-D1 and GNRs-D1/Ang2 were characterized by dynamic light scattering (DLS). The results are shown in Table 1. On the other hand, GNRs-PEG and GNRs-D1/Ang2 were observed using a FEI Inspect F50 scanning transmission electron microscope (STEM). The images are seen in FIGS. 1A and 1B, respectively. GNRs-D1/Ang2 had a transversal hydrodynamic diameter near 4 nm and a longitudinal hydrodynamic diameter near 68 nm. The GNRs showed a zeta potential of −11 mV and a rod shape nanostructure with an aspect ratio (length/width) of approximately 4 (FIG. 2).

TABLE 1
Summary of the transversal and longitudinal hydrodynamic
diameters (Dh) and zeta potentials (pZ) of GNRs-CTAB
and those conjugated to PEG, Ang2, D1, and D1/Ang2.

| | Dh (nm) | | |
|---|---|---|---|
| | Transversal | Longitudinal | pZ (mV) |
| GNRs-CTAB | 2 ± 0.1 | 46 ± 1 | 55 ± 2 |
| GNRs-PEG | 4 ± 0.2 | 56 ± 2 | −23 ± 3 |
| GNRs-Ang2 | 5 ± 0.1 | 62 ± 2 | −17 ± 1 |
| GNRs-D1 | 5 ± 0.1 | 62 ± 1 | −7 ± 6 |
| GNRs-D1/Ang2 | 4 ± 0.2 | 68 ± 6 | −11 ± 1 |

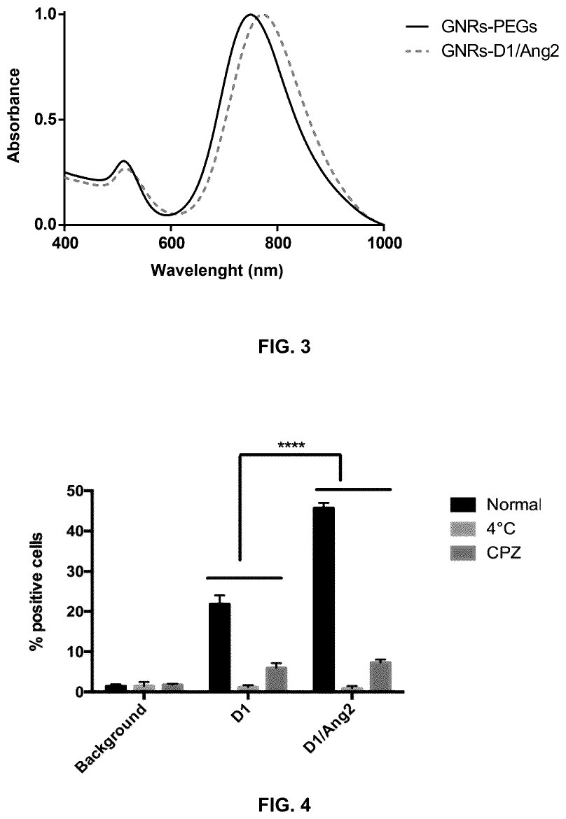

UV-VIS-NIR absorption spectra of GNRs-PEG and GNRs-D1/Ang2 were also analyzed. The spectra were registered at room temperature using a PerkinElmer Lambda 25 spectrophotometer. In the UV-VIS-NIR spectra of GNRs-D1/Ang2, two characteristic peaks of absorption were observed at 520 nm and 760 nm for the transversal and longitudinal plasmons, respectively (FIG. 3).

### Quantification of Peptide Molecules Per GNRs-D1/Ang2

The number of peptide molecules per GNRs-D1/Ang2 was estimated by amino acid analysis and neutron activation analysis. Once the concentration of GNRs by neutron activation analysis was obtained, the number of peptide molecules that were bound to GNRs was determined. The GNRs-peptides solution was lyophilized and hydrolyzed for 72 h in 6 N HCl with a known quantity of aminobutyric acid as a standard. Subsequently, the solution was evaporated under reduced pressure and derivatized for amino acid analysis by HPLC (Morales-Zavala, F., et al. (2017), Nanomedicine, 13, 2341-2350). The number of peptide molecules per GNRs was calculated by dividing the number of peptide molecules per ml of solution by the number of particles per ml of solution. This ratio was calculated in triplicate from three analyses.

The number of peptides per nanoparticle was determined by amino acid analysis and corresponded to 439±23 molecules of D1 and 173±36 molecules of Ang2.

### Example 3. Evaluation of Cell Penetration of GNRs-D1/Ang2

Cell penetration of GNRs-D1/Ang2 into a monolayer of bEnd.3 cells from mouse brain endothelium was evaluated, considering that this is the first step to cross the blood-brain barrier. These cells were incubated with GNRs-D1/Ang2-Alexa647 [0.05 nM] for 2 hours, using GNRs-D1-Alexa647 as a control. The procedure was performed as briefly described below.

### Binding of Alexa Fluor 647 to Conjugated GNRs

When required, conjugated GNRs (GNR-PEG, GNRs-D1, GNRs-Ang2 and GNRs-D1/Ang2) were labeled with the fluorescent probe Alexa Fluor647. Then, 500 μl of conjugated GNRs (20 nM) were washed with Milli-Q water 3 times by centrifugation (20 800 g for 10 minutes) to remove excess free peptide remaining from the conjugation process. Then, GNRs were resuspended in 100 μl of Milli-Q water and 10 μl of Alexa Fluor 647 (ThermoFisher) 1 mg/ml—1 activated with a hydrazide. This group reacts in aqueous solution with carboxylic acids, aldehydes, and ketones. The solution, protected from light, was incubated overnight with mechanical stirring at room temperature and then stored at 4° C. Before use, the excess Alexa Fluor 647 was eliminated by centrifugation.

Evaluation of Cell Penetration of Conjugated GNRs into the bEnd.3 Cell Line by Flow Cytometry

The immortalized bEnd.3 cell line from Mus musculus was used for these experiments. Cells were seeded in 24-well plates at a density of 40 000 cells per well, and after 24 hours of culture they formed a confluent monolayer. The cells were then incubated with GNRs-PEGs, GNRs-D1 and GNRs-D1/Ang2, all Alexa Fluor 647-labeled, at a concentration of 0.05 nM for 2 hours at 37° C. (or 4° C.) (n=5). The concentration of the nanoparticles was similar to that used in previous studies (Morales-Zavala, F., et al. (2017). Nanomedicine, 13, 2341-2350). Subsequently, the cells were washed 3 times, trypsinized, and harvested. Cells were washed once again and flow cytometry analysis was performed using a Beckman Coulter CyAn ADP kit equipped with 3 lasers: 488, 643, and 405 nm. A total of 10 000 cells per sample were evaluated and all assays were performed in triplicate. For the treatment with chlorpromazine (CPZ), the cells were pretreated with CPZ for 15 min at a concentration of 50 μM before incubation with the GNRs (n=6).

The data obtained from the incorporation of GNRs into bEnd.3 cells were analyzed using Turkey's multiple comparisons statistical test.

For the evaluation of cell penetration, three experimental conditions were evaluated:
(i) cells at 37° C. for basal endocytosis,
(ii) cells at 4° C. to inhibit the energy-dependent process; and
(iii) cells pretreated with chlorpromazine (CPZ), an inhibitor of clathrin-dependent endocytosis (Subtil, A. et al. (1994). Cell Sci., 107, 3461-3468).

After these treatments, the cells were harvested and resuspended; the positive cellular association between Alexa-labeled GNRs and the cells was assessed by flow cytometry (FIG. 4). In the basal endocytosis condition with the GNRs-D1-Alexa Fluor 647 treatment, 25% of bEnd.3 cells were positive for fluorescence at 647 nm, whereas 45% of cells were positive when treated with GNRs conjugated to D1 and Ang2, similar to the result described by Velasco-Aguirre et al. (2017) for the incorporation of GNRs-Ang2 in the same cell line. The Ang2 peptide has been described to cross the blood-brain barrier by endocytosis via recognition of the LRP1 receptor, which induces clathrin-dependent endocytosis, followed by transcytosis (Demeule, M. et al. (2008) J. Neurochem., 106(4), 1534-1544). It was evaluated whether this is the route of nanoparticle entry to cells. As shown in FIGS. 1A and 1B, when both GNRs-D1 and GNRs-D1/Ang2 labeled with Alexa647 nanoparticles were incubated with bEnd.3 cells at a low temperature (4° C.) to inhibit energy-dependent processes, these nanorods did not enter to the cells. The percentage of Alexa647-positive population observed at 2 h of incubation decreased to levels comparable to the background, confirming that the cellular incorporation of these nanosystems is an active, energy-dependent process.

In addition, a considerable decrease in the percentage of positive cells was observed in comparison to the normal condition; in the case of GNRs-D1/Ang2, an 84% inhibition was observed when cells were preincubated with CPZ, supporting the hypothesis that GNRs-D1/Ang2 incorporation is clathrin-dependent.

Evaluation of Cell Penetration of GNRs Conjugated to the bEnd.3 Cell Line by TEM

To corroborate cell internalization, TEM was performed for bEnd.3 cells incubated with GNRs-D1/Ang2 at 0.05 nM for 24 hours. The bEnd.3 cells, at 70% confluence, were incubated with the GNRs samples (GNRs-D1/Ang2, 0.05 nM). After 24 hours, the cells were washed with fresh medium and fixed with 2.5% glutaraldehyde in 0.1 M phosphate buffer. Cells were scraped from the surface, collected in an Eppendorf tube, and then allowed to set overnight at 4° C. Then, the pellet was postfixed with 1% OsO₄ in phosphate buffer for 90 min, dehydrated in solutions with acetone gradient and finally embedded in Epon. Sections of 80 nm thickness were cut, placed over copper grids covered with carbon, and subsequently stained with uranyl acetate. Subsequently, micrographs were obtained by TEM (JEOL JEM-1010 microscope).

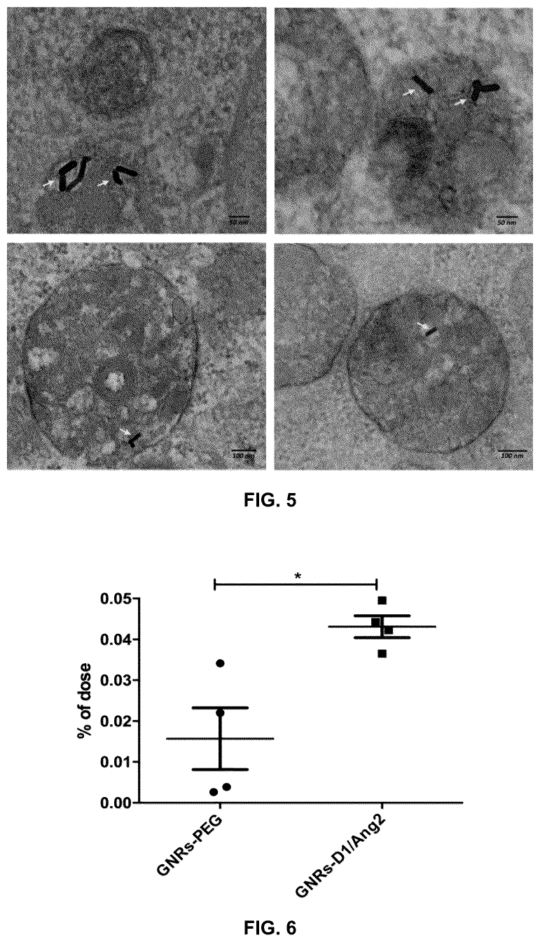

The GNRs were observed inside the cells, mostly within multivesicular bodies, as shown in FIG. 5.

These results are in accordance with an incorporation of the GNRs containing the Ang2 peptide through clathrin-dependent endocytosis, probably mediated by recognition of the LRP1 receptor. The internalization was strongly inhibited by CPZ, which affects the subcellular distribution of components that form the clathrin complex. This result is concordant with the TEM images, where GNRs-D1/Ang2 were observed mainly within multivesicular bodies. LRP1-mediated endocytosis in late stages of internalization directs its ligands to multivesicular bodies. To evaluate the ability of GNRs-Ang2 and GNRs-D1/Ang2 to translocate across the blood-brain barrier (BBB), an in vitro human blood-brain barrier cellular model was used. Hence, human brain capillary endothelial cells from pluripotent stem cells were grown on semi-permeable membranes of a transwell, while co-cultured with bovine pericytes. This BBB model resembles several of the properties of the human BBB and has been used to study the transport of various BBB-shuttles modified with gold nanoparticle. GNRs-Ang2 and GNRs-D1/Ang2 showed an apparent permeability of (1.84±0.69)×10⁻⁷ and (3.18±0.70)×10⁻⁷ cm/s, respectively. Moreover, transmission electron microscopy (TEM) images indicate that GNRs-Ang2 and GNRs-D1/Ang2 were released into the basal compartment (figure not shown), showing that both GNRs were transcytosed.

### Example 4. Evaluation of GNRs-D1/Ang2 in an AD Mouse Model

### Evaluation by Au Quantification

For this study, an AD transgenic mouse model (APPswe/PSEN1dE9, The Jackson Laboratory) was used, which corresponds to a double transgenic that expresses a mouse/human chimera of amyloid precursor protein with the Swedish mutation (Mo/HuAPP695swe) and the mutant human presenilin 1 with the Exon 9 deletion (PS1-dE9), both directed to neurons of the central nervous system (CNS). Both mutations are associated with familial AD. Mice that were 18 months of age were anesthetized intraperitoneally with ketamine xylazine (40 mg/kg and 15 mg/kg, respectively). Then, they were injected intravenously with 100 μl of a 10 nM suspension of GNRs-D1/Ang2 or GNRs-PEG (7 μg of gold per 40 g of body weight, approximately).

The animals were perfused with PBS, and the organs of interest were obtained, frozen in liquid nitrogen, and lyophilized at −50° C. under a pressure of 0.137 mbar. Once lyophilized, the dried organs were ground in a porcelain crucible to homogenize the samples. Once ground, they were placed in an oven at 120° C. until reaching a constant weight. Finally, their gold content was quantified. The experiments were performed four times in independent experiments.

FIG. 6 shows the percentage of the dose reaching the brain; for control GNRs-PEGs the percentage was 0.0157±0.0151%, while for the GNRs-D1/Ang2 it was 0.043±0.005%, significantly higher than the control. This result indicates that GNRs-D1/Ang2 GNRs reaches the brain almost three times more than GNRs-PEGs, which agrees with the expected results according to the rational design employed and the data reported by Velasco-Aguirre et al. (2017), who described that the functionalization of GNRs with Ang2 improves the delivery of GNRs to the Central Nervous System (CNS), in comparison to GNRs-PEG in a healthy rat model.

In order to determine whether GNRs-D1/Ang2 were localized in the amyloid plaques present in the brain, a gold enhancement protocol with hematoxylin and Congo red staining was also used. Congo red allows the detection of amyloid plaques.

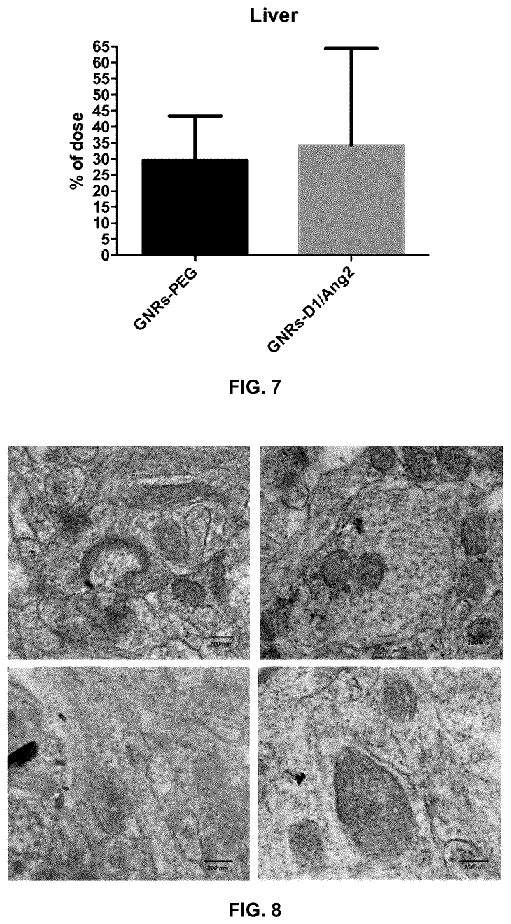

The gold content in the liver was also analyzed and it was observed that there were no significant differences between the control GNRs-PEGs and GNRs-D1/Ang2, with retention values close to 30% of the dose for both groups, as shown in FIG. 7.

### Evaluation by Scanning Transmission Electronic Microscopy (STEM)

To confirm the presence of GNRs in the AD mice brains, STEM was used.

The animals treated with GNRs-D1/Ang2 were perfused with PBS as described in the previous example, and then perfused with a solution of 2% paraformaldehyde with 2.5% glutaraldehyde in 0.1 M sodium phosphate buffer. Then, the brains were removed, and the samples were processed and observed by dark-field scanning transmission electron microscopy.

The presence of GNRs-D1/Ang2 in the hippocampal parenchyma was confirmed by STEM (FIG. 8).

### Evaluation by Fluorescence

To evaluate the targeting of GNRs-D1/Ang2 into the CNS, the nanosystem was labeled with Alexa Fluor 647, and the Alexa Fluor 647-GNRs-D1/Ang2 were administered to AD transgenic mice (n=2). As control, Alexa Fluor 647-GNRs-PEG were also administered (n=1). The treatment was performed as described in the previous example.

After finishing treatment, the brains were fixed by immersion in a 4% paraformaldehyde solution in PBS for 24 hours at room temperature. Once fixed, the brains were preserved in a solution of 30% sucrose and stored at 4° C. until use.

Using a cryostat, brain slices (30 μm) were obtained. The selected slices were permeabilized with 0.4% TBS-Triton X-100, and then blocked with 0.15 M glycine for 15 minutes, 3 times with NaBH₄ (10 mg/ml) for 10 minutes, 50 mM ammonium chloride for 10 minutes and finally, with PBS 1×-Triton 0.2%-BSA 5% for 1 hour. The slices were incubated for 24 hours with WO2 antibody (1:200 dilution in blocking solution), which selectively labels the amyloid plaques. Then, the tissues were washed 4 times for 10 minutes each with PBS and incubated with the secondary antibody, mouse anti-IgG conjugated to Alexa Fluor 488 (1:2000 in blocking solution), for 1 hour at room temperature.

Finally, images were obtained using a Carl Zeiss microscope model Axio Imager A1, equipped with a Carl Zeiss color CCD camera model AxioCam MRc5. For fluorescence microscopy, a mercury lamp (Zeiss HBO 100) was used with the following sets of filters (green: Zeiss filter set 09, excitation: 450-490, emission: LP 515, and red: Zeiss filter set 45, excitation: 560/40, emission: 630/75). Carl Zeiss software AxioVision version 4.8 was used for the acquisition of images.

An exposure time of 400 ms was used for the green channel and 1000 ms for the red channel.

Prior to quantifying the obtained images, contrast adjustments and background subtractions were applied using ImageJ-FIJI software to improve automated segmentation.

Segmentation and data collection were performed using CellProfiler v.2.0, according to the following steps: image segmentation for both GNRs-D1/Ang2 associated labels and amyloid plaque markers, measurement of correlation between both images, evaluation of the relationship between segmented labels, classification of objects as overlapped or non-overlapped according to the relation of the specific label to amyloid plaques, masking of labels to calculate the region of GNRs-D1/Ang2 specific labeling that overlapped with amyloid, measurement of the area occupied by the overlapped region, and calculation of the overlap between labels as a percentage of the total amyloid plaque area. For each treated brain, at least 6 slices were obtained independently for each analysis.

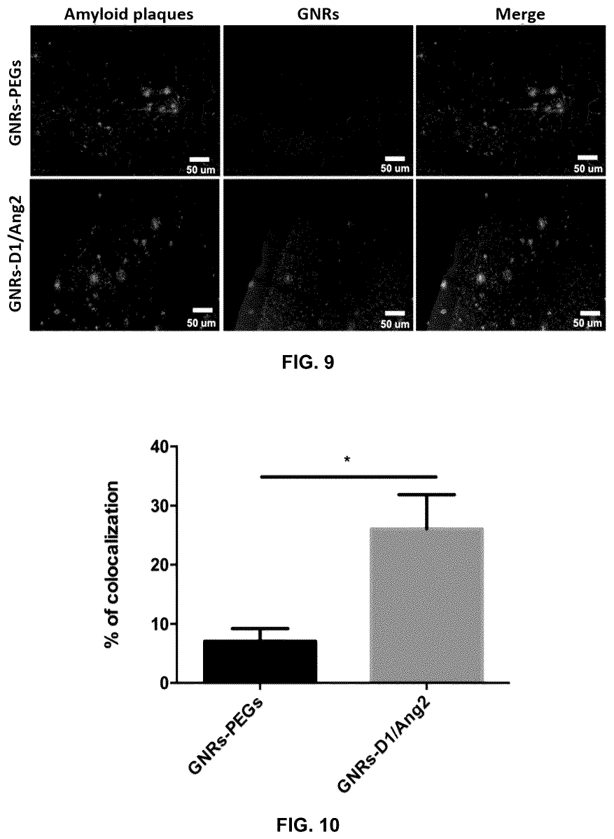

Using Alexa Fluor 647-labeled GNRs as a control and GNRs-D1/Ang2, the localization of GNRs in brain slices of AD mice and their distribution with respect to amyloid plaques was then evaluated. The GNRs-D1/Ang2 labeled with Alexa Fluor 647 were administered intravenously. After 15 minutes, the mice were anesthetized, the brains were removed and fixed, and histological sections were obtained. The amyloid plaques were labeled by immunohistochemistry with WO2 antibodies and visualized in green (Alexa Fluor 488). A clear Alexa Fluor 647 signal (red) was observed in AD mice injected with GNRs-D1/Ang2-Alexa Fluor 647, which was concordant with the distribution of the WO2 green signal corresponding to amyloid plaques, whereas the AD mice injected with GNRs-PEGs-Alexa Fluor 647 showed a background red signal that was not coincident with amyloid plaques (FIG. 9). The overlapping signals were evaluated with the ImageJ-FIJI and Cell Profiler software, and the percentage of colocalization between the marks associated with GNRs-D1/Ang2 and the amyloid plaques was 22% (FIG. 10). For the GNRs-PEGs control, the percentage of overlapping signal was only 7%.

### Example 5. Effect of Recurrent Treatment with GNRs-D1/Ang2 in the AD Mouse Model

Twelve-month-old APPswe/PSEN1dE9 mice (AD mice) that had free access to food and water were used and maintained on a 12 h dark/light cycle in a room with controlled temperature (25±2° C.).

The mice were divided into five groups:
1) free D1 peptide (n=2),
2) GNRs-PEG (n=1),
3) GNRs-D1 (n=2),
4) GNRs-Ang2 (n=4), and
5)

$$GNRs - D 1 / Ang 2 ( n = 4 ) .$$

Mice received intraperitoneal (i.p.) injections of 10 nM GNRs (groups 2-5) or free D1 peptide (group 1) in approximately 100 μl (approximately 10 μg of gold per 30 g of body weight) every two days for 30 days, giving a total of 15 administrations. The body weight of animals and their behaviors were carefully recorded daily during the extension of the experiment. One day after the last injection, mice were sacrificed, and the brains were immediately collected.

Once it was determined that the GNRs-D1/Ang2 nanosystem reached the CNS and located with the amyloid plaques, it was evaluated whether the GNRs-D1/Ang2 conjugate—where the D1 peptide recognizes and disaggregates amyloid aggregates—affected amyloid load and inflammatory markers in AD mice. To this end, the AD mice were treated with GNRs-D1/Ang2 and GNRs-Ang2 as a control (100 μl, 10 nM) every two days, giving a total of 15 administrations in a period of 30 days, after which the animals were anesthetized and perfused with PBS. The brains were extracted to evaluate the number of plaques and amyloid loads using Thioflavin-S (Th-S), and GFAP (Glial Fibrillary Acidic Protein) was used to follow reactive astrocytes in the brain histological sections.

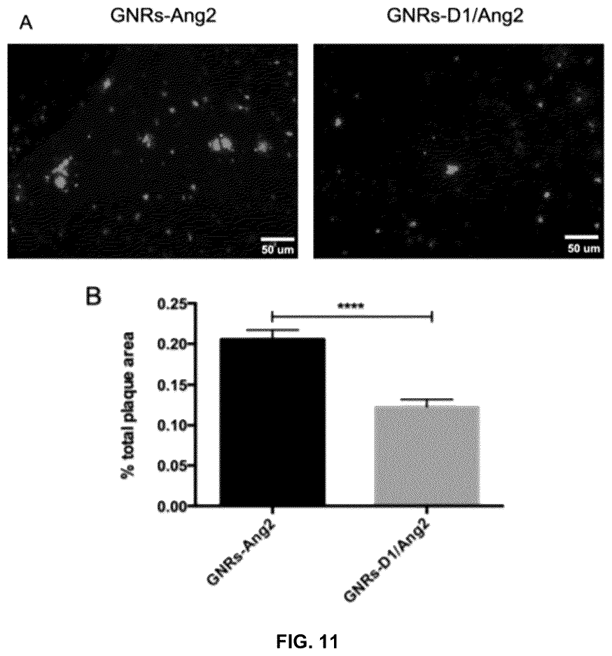

The AD mice injected with GNRs-D1/Ang2 showed fewer and smaller amyloid plaques than those injected with control GNRs-Ang2 (FIG. 11A). Using the ImageJ and CellProfiler software, the number, area, and intensity of the Th-S signal associated with amyloid plaques and the percentage of plaque area with respect to total area in the GNRs-Ang2-treated control group and in the GNRs-D1/Ang2-treated group (FIG. 11B) were quantified. In the experimental group, the percentage of the area occupied by amyloid plaques in the cortex was lower than that of the control group.

### Example 6. Evaluation of Amyloid Load and Inflammation in AD Mice Treated with GNRs-D1/Ang2

The brains obtained from the experimental groups mentioned in Example 5, were fixed and the brain slices were manipulated as mentioned in the following examples.

### Immunolabeling

Healthy mice (WT) and AD mice (APPswe/PSEN1dE9) were sacrificed, the brains were removed and fixed, and the samples were prepared as described in Example 3.

Immunolabeling was performed with GFAP (1:500, Cell Signaling). The antibodies anti-mouse-Alexa Fluor-488 and anti-rabbit-Alexa Fluor 555 (1:1000, Molecular Probes) were used as secondary antibodies (Estrada, L. D. et al. (2016). Alzheimers Dis., 54, 1193-1205).

### Th-S Staining Protocol

Th-S staining was performed as previously described (Chacón, M. A. et al., (2004), Mol. Psychiatry, 9, 953-961).

Briefly, brain slices of AD mice were dehydrated and rehydrated with ethanol and xylol batteries, then, the slices were incubated in distilled water for 10 min. Then, the slides were immersed in the Th-S solution (0.1% Th-S in 70% ethanol) for 5 min and washed twice in 70% ethanol for 30 s and 3 times in distilled water for 2 min. Following these washes, the sections were mounted on gelatin-coated slides and coverslipped with Dako mounting medium in the dark.

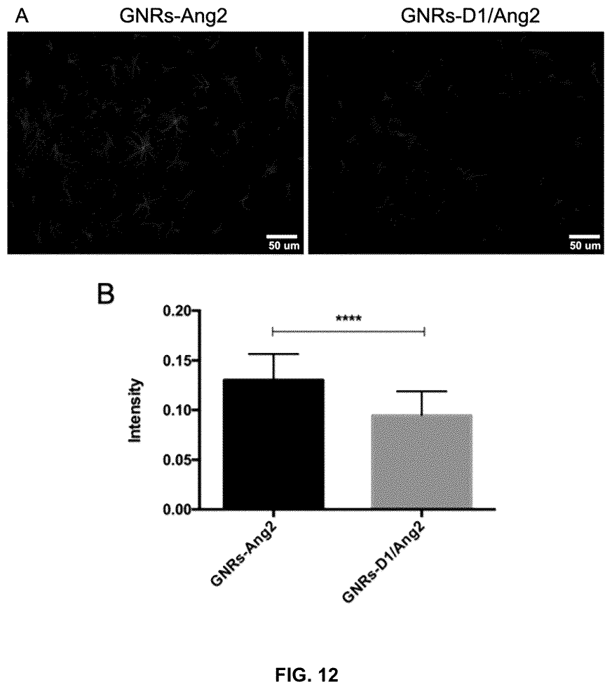

The GFAP signal for astrocytes was evaluated as a mark of brain inflammation and FIG. 12A shows that GFAP intensity was higher in the cortex of GNRs-Ang2-treated AD mice, compared to GNRs-D1/Ang2-treated AD mice. According to the quantification of the GFAP signal, lower levels of active astrocytes were found in AD mice treated with GNRs-D1/Ang2 (FIG. 12B).

These results of recurrent treatment with GNRs-D1/Ang2 and evaluation of amyloid load and inflammation in AD mice agree with the design of the nanosystem that is the object of the invention as a theragnostic tool. GNRs-D1/Ang2 were specifically targeted to amyloid plaques and served as a drug delivery device for D1, reducing the Aβ load. In this case, the control group treated with GNRs-Ang2 did not have the D1 therapeutic peptide, but the Ang2 peptide that improves delivery to the brain; this group presented a higher number of amyloid plaques and a major area of the cortex occupied by the amyloid plaques, compared to the experimental group treated with GNRs-D1/Ang2. Similarly, the experimental group presented lower inflammation in the cortex, which correlated well with the decrease of the amyloid load in this region of the brain. The activity of a small quantity of nanoparticles that reach the brain can be attributed to the local accumulation of the cargo (the D1 peptide) associated with the nanoparticle that produces an efficient effect.

It is important to note that, after the chronic treatment, the animals did not show significant indicatives of distress, toxicity, pain, or discomfort, such as irregular respiration, loss of hair, ataxia or other motor disabilities, nor death.

### Example 7. In Vivo Evaluation of GNRs-D1/Ang2 in the AD Mouse Model by Micro-CT Analysis

To evaluate the scope of conjugated GNRs as a contrast agents for micro-CT, at the end of the different administrations or treatments with said GNRs, the animals were scanned using a micro-CT. The animals were kept under sedation during the scanning procedure using isoflurane.

In vivo tomographies of the brains were performed using a micro-CT Scanner (Bruker, Skyscan model 1278) with a nominal resolution value of 51 μm, without aluminum filter, and with a tube voltage of 39 kV. The reconstruction was done using the SkyScanNRecon software accelerated by GPU. Ring artifact reduction, Gaussian smoothing (2%), and beam hardening correction (23%) were applied. Volume rendered 3D images were generated using the CT-Voxel (“CT-Vox”) software. The experiments were carried out with n=3 for the chronic treatment and n=1 for the acute treatment.

Taking advantage of the high X-ray attenuation coefficient of gold (Betzer, O. et al. ACS Nano, 2017, 11, 10883-10893), the presence of GNRs in the CNS in vivo was determined by the micro-CT technique, considering the possibility of applying the nanosystem of the invention as an in vivo diagnostic method for AD.

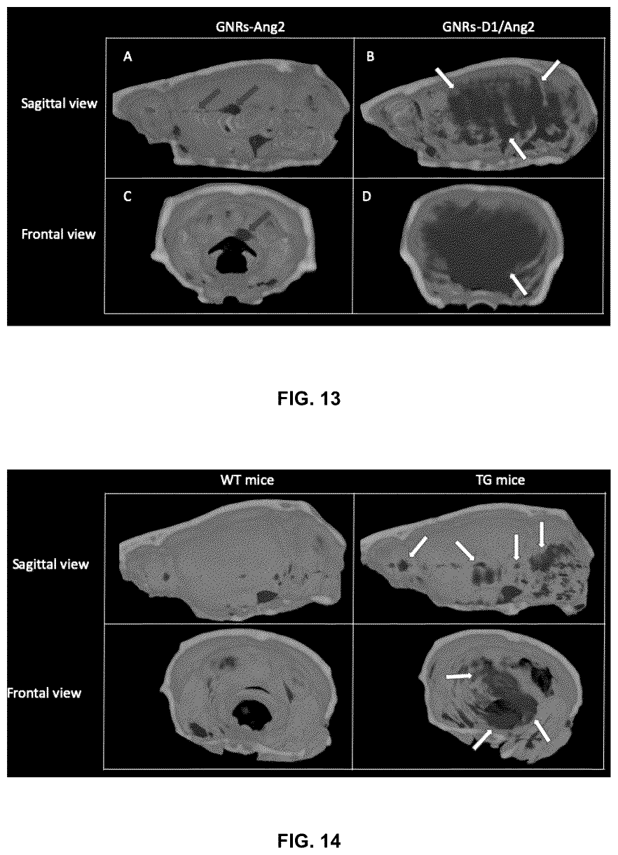

Before being sacrificed, the AD mice treated with GNRs-Ang2 and GNRs-D1/Ang2 through 15 injections over 30 days were analyzed by micro-CT. The micro-CT signals were higher in animals treated with GNRs-D1/Ang2 than in those treated with GNRs-Ang2, as shown in FIG. 9. FIGS. 13A and 13C correspond to the animals treated with GNRs-Ang2, in the sagittal and frontal sections, respectively. The signal associated with the GNRs was located only in the center of the brain, in a straight line that crossed the brain, which could be attributed to the nanoparticles present in the circulation. On the other hand, in the animals treated with GNRs-D1/Ang2 (FIGS. 13B and 13D), the signals associated with the GNRs were observed in different brain regions, which are related to the accumulation of amyloid plaques in this model in vivo (Ordoñez-Gutierrez, L. et al. (2016) J. Alzheimer's Dis., 54, 645-656).

This difference supports the notion that the presence of the D1 peptide in the nanosystem allows its retention in the brain of AD mice. This result is in agreement with the rational design of the nanosystem of the invention, as the D1 peptide is essential for the capacity to recognize the Aβ aggregates present in the brain with AD.

Finally, to determine whether a single dose of the nanosystem allowed the detection of the presence of GNRs-D1/Ang2 associated with amyloid aggregates in the brain, differences in the GNR signal between healthy and AD animals were evaluated. For this purpose, micro-computed tomography (micro-CT) scans of the AD mice (TG in FIG. 14) and healthy mice (WT in FIG. 14) that were treated with GNRs-D1/Ang2 for 15 min were performed to assess whether the presence of the nanosystem in the brain of the AD mice could be detected with a single dose and whether there were differences in this signal, compared with healthy animals. As shown in FIG. 14, a higher signal associated with GNRs-D1/Ang2 was detected in the brain of AD mice (TG) compared to the signal in the healthy mice (WT).

In addition, a high signal was observed in the cerebellum of the TG mouse. According to Ordoñez-Gutierrez et al., 2016, β-amyloid aggregates in the cerebellum of this transgenic AD mouse model, and therefore the present results, support the early interaction of circulating GNRs-D1/Ang2 with Aβ deposits.

In the case of animals treated with GNRs-PEG, only one signal associated with said GNRs-PEG was detected in the center of the brain, forming a straight line through it, very similar to that observed in control animals treated with GNRs-Ang2 for 30 days, which could be related to the GNRs in circulation. For WT mice treated with a single dose of GNRs-PEG or without treatment, no signals associated with the presence of GNRs in the brain were detected.

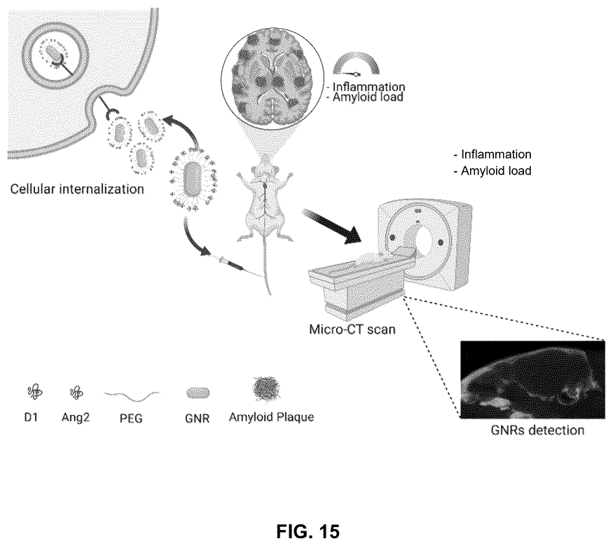

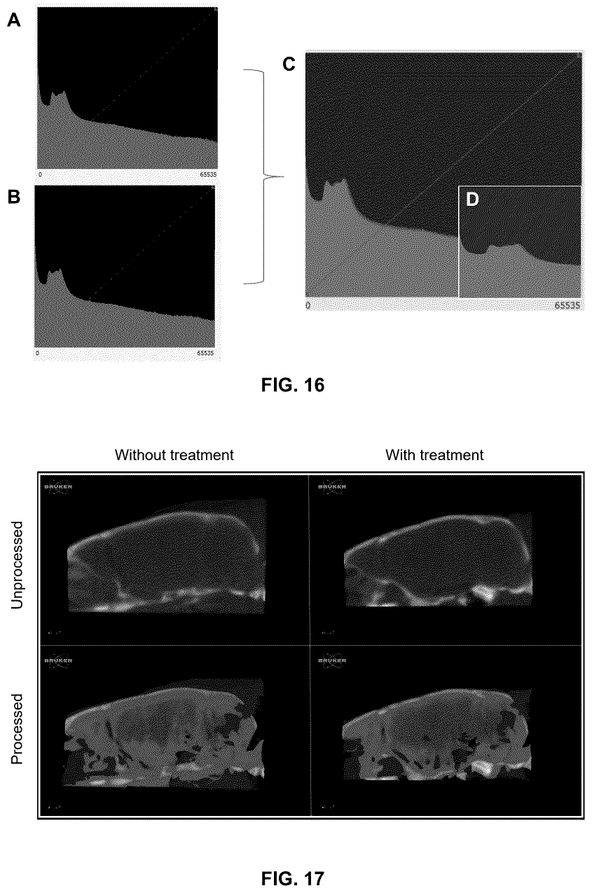

From all the studies performed, it is concluded that an in vivo therapeutic peptide delivery system based on GNRs, with theranostic potential for application in AD subjects, and compatible with the micro-CT methodology, has been developed. A schematic of this procedure is shown in FIG. 15. The GNRs conjugated to D1 and Ang2 peptides induced the diminution of both the amyloid load and inflammatory markers in the brain of the AD murine model by using a micro-CT. Moreover, differences in the in vivo detection of GNRs-D1/Ang2 were observed between healthy and AD mice, when both the brains of animals treated for one month and those treated with a single dose of the nanosystem were analyzed. It was demonstrated that the GNRs conjugated to the two peptides (D1 and Ang2) improved the delivery and retention of this nanosystem in the brain, reinforcing the therapeutic benefits associated with the β sheet breaker ability of the D1 peptide.

### Example 8. Evaluation of GNRs-D1/Ang2 Dose

This experiment was designed to determine the dose range of GNRs-D1/Ang2 and its possible effect on the detection and visualization of amyloid aggregates in Alzheimer's disease transgenic mice (TG) by micro-CT. For this purpose, a dose 10 times more concentrated and a dose 10 times more diluted of GNRs-D1/Ang2 than the used in previous in vivo experiments was used.

Three 11-month-old APPswe/PSEN1dE9 transgenic mice (TG) with Alzheimer's disease were used. The brains of the mice were scanned by micro-CT in two stages: first, a pre-treatment stage to obtain the basal images of the animal, and a post-treatment stage to obtain the images with GNRs-D1/Ang2. In this way, the animal was compared before and after the administration of the GNRs-D1/Ang2 and the observed changes were evaluated. The animals were administered doses of 1, 10, and 100 nM of GNRs-D1/Ang2, equivalent to 8.6, 86, and 860 μg of Au per kg of body weight.

### 1 nM Dose

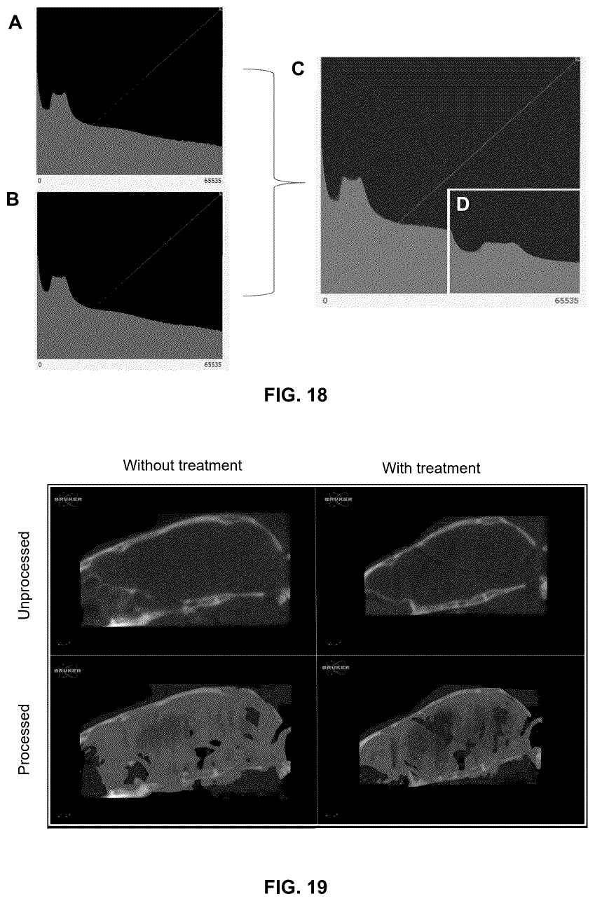

The image histograms of the different stages were analyzed and a shift of pixels toward higher intensity values was observed (FIGS. 16A-D), which is reflected in an increase in shades of gray within the cranial vault, which is the area of interest. This change can be perceived when observing the image directly (unprocessed) or after processing (FIG. 17) where the change produced by the administration of the GNRs-D1/Ang2 is clearly more visible, showing a larger area of grays along the vault in the shape of clouds which are larger than those seen in the pre-treatment stage.

### 10 nM Dose

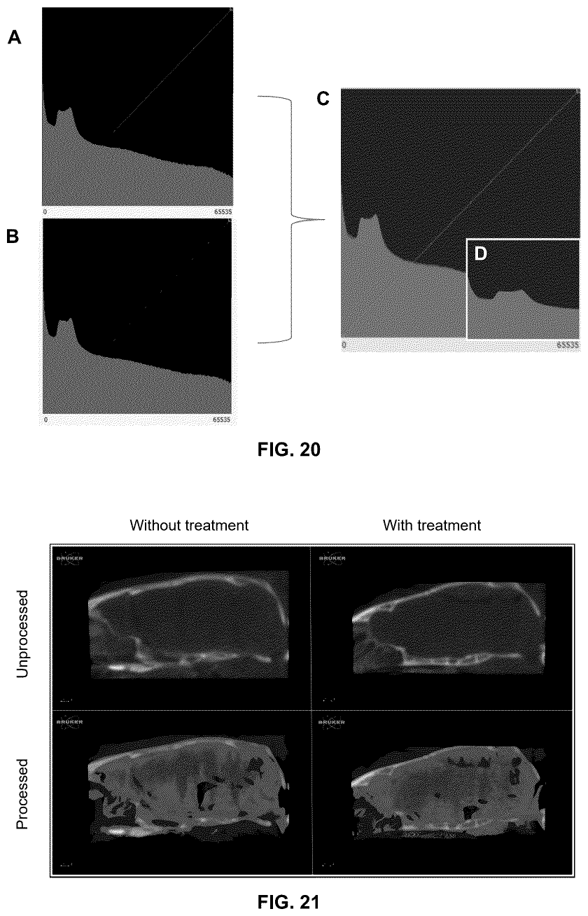

The image histograms of the different stages were analyzed and a change in the intensities of the pixels of the image was observed, showing an increase in the number of pixels with a similar intensity value in the area of interest (FIGS. 18A-D), which is reflected in an increase in the shades of gray within the cranial vault, which is the area of interest. This change can be perceived when observing the image directly (unprocessed) or after processing (FIG. 19) where the change produced by the administration of the GNRs-D1/Ang2 is clearly more visible, showing a larger area of grays along the vault in the shape of clouds which are larger than those seen in the pre-treatment stage.

### 100 nM Dose

The image histograms of the different stages were analyzed and a slight change in the intensities of the pixels of the image was observed (FIG. 20A-D), which is lower than those observed in the treatments with 1 and 10 nM. When reviewing the unprocessed 3D images of the animal (FIG. 21), it is observed that the shades of gray are more homogeneously distributed after treatment. When looking at the processed images (FIG. 21), a change in the distribution of grays can be clearly seen, which are more homogeneously distributed after the treatment with the GNRs-D1/Ang2, but in terms of intensity level the change is much smaller than that observed with the other two doses.

From these experiments it was concluded that in the three doses used it was possible to appreciate changes between the pre-treatment and post-treatment stages, so that with the range of concentrations used it is feasible to perceive the effect of the GNRs-D1/Ang2 and their visualization by micro-CT. With the 1 and 10 nM doses it is possible to visualize both a change in the distribution grays and a change in intensity (histograms and images), but in the case of the 100 nM dose a change in distribution (image), and not so much in intensity (histogram), is more noticeable. Therefore, the dose range preferably used in the present invention is within the range where changes both in distribution and intensity between the different stages of the treatment are perceived.

1. A reagent that binds to β-amyloid plaques in a subject, comprising gold nanorods conjugated to D1 and Angiopep-2 peptides, CHARACTERIZED in that it includes a dose of said gold nanorods conjugated to D1 and Angiopep-2 peptides between about 8.6 and 860 μg of Au per kg of body weight of the subject, and a pharmaceutically acceptable vehicle.

2. The reagent according to claim 1, CHARACTERIZED in that the gold nanorods are conjugated to the D1 and Angiopep-2 peptides at a molar ratio of gold nanorods:peptides of 1:600.

3. The reagent according to claim 1, CHARACTERIZED in that the D1 and Angiopep-2 peptides have a molar ratio of 1:9.

4. The reagent according to claim 1, CHARACTERIZED in that the nanorods have all their dimensions in the nanometer range and have a length/width aspect ratio between 1 and 5.

5. The reagent according to any one of the preceding claims, CHARACTERIZED in that the reagent is formulated for intranasal or intravenous administration.

6. A method for targeting a reagent including gold nanorods conjugated to D1 and Angiopep-2 peptides to β-amyloid plaques in a subject, CHARACTERIZED in that it comprises contacting said reagent at a dose between 8.6 and 860 μg of Au per kg of body weight of the subject with a tissue or organ of the subject, and allowing said reagent to bind to the β-amyloid plaques present in the tissue or organ.

7. The method for targeting the reagent according to claim 6, CHARACTERIZED in that the organ is the brain.

8. A method for detecting β-amyloid plaques in a subject, using a reagent including gold nanorods conjugated to D1 and Angiopep-2 peptides, CHARACTERIZED in that it comprises the steps of:
a) administering to the subject a dose of said reagent between about 8.6 to 860 μg of Au per kg of body weight of the subject;
b) allowing said reagent to bind to the β-amyloid plaques present in a tissue or organ of said subject; and
c) subjecting the subject to an imaging study to detect the β-amyloid plaques to which said reagent binds.

9. The method for detecting β-amyloid plaques according to claim 8, CHARACTERIZED in that it comprises administering the reagent to the subject intravenously or nasally.

10. The method for detecting β-amyloid plaques according to claim 8, CHARACTERIZED in that the organ is the brain.

11. The method for detecting β-amyloid plaques according to claim 8, CHARACTERIZED in that the imaging study is by computed tomography.

12. A method for evaluating the progression of the accumulation of β-amyloid plaque over time in a subject, using a reagent including gold nanorods conjugated to D1 and Angiopep-2 peptides, CHARACTERIZED in that it comprises the steps of:
a) determining a reference value of the number of β-amyloid plaque present in a tissue or organ of said subject at a time 0, said determination comprising the steps of:
i) administering to the subject a dose of said reagent between about 8.6 and 860 μg of Au per kg of body weight of the subject;
ii) allowing the reagent to bind to the β-amyloid plaques present in the tissue or organ of said subject; and
iii) subjecting the subject to an imaging study to quantify the β-amyloid plaques to which said reagent binds;
b) determining a value for the number of β-amyloid plaque present in the tissue or organ of said subject at a time n, said determination comprising steps (i) through (iii);
c) comparing the value of the number of β-amyloid plaque at the time n with the reference value at the time 0; and
d) if the value of the number of β-amyloid plaques at the time n is greater than the reference value at the time 0, it is an indication of the progression of the accumulation of β-amyloid plaques over time in the subject.

13. The method for evaluating the progression of the accumulation of β-amyloid plaque according to claim 12, CHARACTERIZED in that it comprises administering the reagent to the subject intravenously or nasally.

14. The method for evaluating the progression of the accumulation of β-amyloid plaque according to claim 12, CHARACTERIZED in that the organ is the brain.

15. The method for evaluating the progression of the accumulation of β-amyloid plaque according to claim 12, CHARACTERIZED in that the imaging study is by computed tomography.

16. A pharmaceutical composition for treating neurodegenerative diseases associated with β-amyloid plaques in a subject, comprising gold nanorods conjugated to D1 and Angiopep-2 peptides, CHARACTERIZED in that it includes a dose of said gold nanorods conjugated to D1 and Angiopep-2 peptides between about 8.6 and 860 μg of Au per kg of body weight of the subject, and a pharmaceutically acceptable vehicle.

17. The pharmaceutical composition according to claim 17, CHARACTERIZED in that the gold nanorods are conjugated to the D1 and Angiopep-2 peptides at a molar ratio of gold nanorods:peptides of 1:600.

18. The pharmaceutical composition according to claim 17, CHARACTERIZED in that the D1 and Angiopep-2 peptides have a molar ratio of 1:9.

19. The pharmaceutical composition according to claim 17, CHARACTERIZED in that the nanorods have all their dimensions in the nanometer range and have a length/width aspect ratio between 1 and 5.

20. The pharmaceutical composition according to any one of the preceding claims, CHARACTERIZED in that the reagent is formulated for intranasal or intravenous administration.

21. The pharmaceutical composition according to any one of the preceding claims, CHARACTERIZED in that the neurodegenerative disease associated with β-amyloid plaques is Alzheimer's disease.

22. A method for treating diseases associated with β-amyloid plaques in a subject using a pharmaceutical composition including gold nanorods conjugated to D1 and Angiopep-2 peptides, CHARACTERIZED in that it comprises administering to the subject a dose of said pharmaceutical composition between about 8.6 and 860 μg of Au per kg of body weight of the subject.

23. The method of treatment according to claim 22, CHARACTERIZED in that it comprises administering the pharmaceutical composition to the subject intravenously or nasally.

24. The method of treatment according to claim 22, CHARACTERIZED in that the neurodegenerative disease associated with β-amyloid plaques is Alzheimer's disease.


SEQUENCE LISTING (2 sequences)

SEQ ID NO: 1 - protein, 19 aa, Homo sapiens
TFFYGGSRGKRNNFKTEEY

SEQ ID NO: 2 - protein, 12 aa, Homo sapiens
QSHYRHISPAQV